(19) United States
(12) Patent Application Publication
(10) Pub. No.: US 2025/0345789 A1
(43) Pub. Date: Nov. 13, 2025

(54) GEOMETRICALLY ENHANCED LATERAL FLOW IMMUNOASSAYS FOR QUANTITATIVE ASSESSMENT OF BIOMARKERS IN POINT-OF-NEED APPLICATIONS

(71) Applicant: 1469694 B.C. Ltd., Surrey (CA)

(72) Inventors: Razieh Salahandish, Toronto (CA); Fatemeh Haghayegh, Toronto (CA); Ashissh Raichura, Vancouver (CA); Elnaz Haghani, Toronto (CA); Alireza Norouziazad, Toronto (CA)

(21) Appl. No.: 19/200,132

(22) Filed: May 6, 2025

Related U.S. Application Data
(60) Provisional application No. 63/643,523, filed on May 7, 2024.

(51) Int. Cl. B01L 3/00; G16H 10/40

(52) U.S. Cl. CPC B01L 3/5023; G16H 10/40; B01L 2200/0689; B01L 2200/16; B01L 2300/04; B01L 2300/0672; B01L 2300/069; B01L 2300/0825; B01L 2400/0406

(57) ABSTRACT

Lateral flow immunoassay systems include test strips having unique geometries to enhance fluid dynamics, including but not limited to: (1) conjugate pads having sinusoidal, meandering, and nozzle-diffuser patterns; and (2) nitrocellulose membrane pads having constriction zones corresponding to the location of T-lines and/or C-lines. The systems can further include a cartridge with a two-step rotation mechanism for contamination-free sample delivery to the sample pad of a test strip, and an AI-based detection system for quantifying the concentration of a detected analyte based on colorimetric signal intensities. The system exhibits improved analyte sensitivity over prior systems, reduces traditional assay time, eliminates subjective interpretation of colorimetric signal intensities, and quantifies analyte concentration levels.

### CROSS-REFERENCE TO RELATED APPLICATIONS

This application claims the benefit of U.S. Provisional Patent Application No. 63/643,523, filed on May 7, 2024, and titled “Geometrically Enhanced Lateral Flow Immunoassays for Quantitative Assessment of Biomarkers in Point-of-Need Applications,” the disclosure of which is incorporated herein by reference in its entirety.

### BACKGROUND OF THE INVENTION

Traditionally, lateral flow immunoassays (LFIAs) are diagnostic tests used to detect the presence or absence of a target substance, such as antigens, antibodies, or other biomarkers, in a liquid sample without the need for specialized equipment. They are commonly used in medical diagnostics (e.g., pregnancy tests, COVID-19 antigen tests) and other fields like food safety or environmental testing.

LFIAs have become a cornerstone of point-of-care diagnostics due to their simplicity, low cost, and rapid results, making them ideal for applications such as infectious disease screening, pregnancy testing, and drug detection. Traditional LFIA test strips consist of a sample pad, a conjugate pad with labeled recognition elements (e.g., antibodies conjugated to gold nanoparticles), a nitrocellulose membrane with test and control lines, and a wicking pad that drives capillary flow. These strips typically operate in sandwich or competitive assay formats, producing a colorimetric signal visible to the naked eye or through basic imaging. Their ease of use and minimal equipment requirements align with the World Health Organization's ASSURED criteria (affordable, sensitive, specific, user-friendly, rapid, robust, equipment-free, and accessible), positioning LFIAs as a powerful tool for decentralized healthcare, particularly in resource-limited settings.

Despite their widespread use, conventional LFIAs suffer from significant limitations, particularly in sensitivity and quantification. Most LFIAs are designed for qualitative or semi-quantitative detection, indicating only the presence or absence of a target analyte, which restricts their utility in early disease diagnosis where low biomarker concentrations are critical. For instance, the relatively low sensitivity of standard LFIAs (e.g., limit of detection [LOD] of 1.78±0.08 ng/mL for hepatitis B surface antigen) limits their ability to detect trace analytes, such as those present in early-stage infections or chronic conditions. Additionally, the lack of effective solutions for quantifying signal intensity hinders their application in precision medicine, where accurate measurement of biomarker concentrations is essential for monitoring disease progression or therapeutic efficacy.

Another challenge is the limited capability of conventional LFIAs for multiplexing, or the simultaneous detection of multiple biomarkers. Current LFIA designs typically focus on single-analyte detection, with test strips configured for one target per assay. Multiplexing, particularly in commercial and clinical settings, remains underexplored due to challenges in integrating multiple test lines without compromising signal clarity or increasing assay complexity. This limitation is particularly pronounced in point-of-care applications requiring comprehensive diagnostic panels, such as those for infectious diseases, cardiovascular markers, or metabolic disorders.

Sample collection and processing, especially for finger-prick blood, pose additional hurdles for LFIA-based diagnostics, particularly in self-testing scenarios. Traditional LFIA kits require multiple manual steps, including sample collection, dilution with running buffers, and application to the test strip, which can be error-prone for untrained users. Proper mixing of blood with buffers is critical for reproducible results, yet existing systems often rely on complex accessories or external tools, increasing the risk of user error and reducing accessibility. Microfluidic platforms with multiple valves have been proposed to streamline sample handling, but their high cost and manufacturing complexity make them impractical for large-scale production or widespread adoption in home settings.

Recent advancements in LFIA technology have attempted to address these challenges through various approaches, such as fluidic pathway modifications, assistive technologies (e.g., electrophoresis), and geometric changes to enhance test line intensity. For example, constriction shapes and laser-induced micromixers have been explored to improve antigen-antibody interactions. However, these solutions often compromise assay timing, increase background noise, or require sophisticated equipment, undermining the ASSURED criteria. Moreover, while AI-based image analysis has been applied in other diagnostic fields, its integration with LFIAs for automated, quantitative signal acquisition remains limited. A need therefore exists for a platform that combines enhanced sensitivity, multiplexing, automated sample processing, and AI-driven quantification to enable reliable, user-friendly diagnostics for both clinical and at-home use.

### SUMMARY OF THE INVENTION

The systems and methods described herein address the shortcomings of prior solutions by introducing a geometrically enhanced LFIA platform that can be used, optionally, with a new finger-prick blood collection cartridge and AI-based detection. The systems and methods described herein offer significant improvements in sensitivity, quantification, and ease of use compared to traditional LFIA test strips and blood collection cartridges. More particularly, the disclosed embodiments can pertain to systems, devices, and methods for quantitatively detecting biomarkers in biological samples using geometrically enhanced LFIA test strips integrated with microfluidic components, automated sample processing cartridges, and artificial intelligence (AI)-based detection systems. The embodiments address the need for sensitive, user-friendly, and multiplexed diagnostic platforms capable of detecting a wide range of analytes, including proteins, peptides, small molecules, and minerals, in various body fluids such as blood, saliva, urine, sweat, and tears. The embodiments include novel test strip geometries, microfluidic integration, and a cartridge with a two-step rotation mechanism for automated sample processing, enabling sensitive, reliable, and user-friendly point-of-care diagnostics.

In one aspect, devices described herein comprise a lateral flow test strip with one or both of a geometrically modified conjugate pad and nitrocellulose membrane, featuring non-linear patterns such as constriction zones, serpentine, sinusoidal, meandering, spiral, and/or nozzle-diffuser designs to enhance mixing and flow control. As used herein, the term “non-linear,” with respect to test strips or any component thereof, is intended to mean any shape whose lateral sides and/or central or medial axis (along the shape's longitudinal extension) is non-linear. The test strip can be housed within a cartridge with a capillary blood collection chip and a two-step rotation capsule for automated sample-to-result processing.

In another aspect, systems described herein comprise the test strip, a cartridge with a two-step rotation mechanism, and an AI-based detection module. The AI module can analyze colorimetric signals from test line intensities using machine learning to quantify biomarker concentrations, achieving a limit of detection (LOD) as low as 0.55±0.04 ng/mL for hepatitis B antigens, as just an example.

In a further aspect, methods described herein include detecting biomarkers using the test strip and cartridge, and can involve collecting a finger-prick blood sample, mixing it with a buffer via a two-step rotation, and quantifying biomarker concentrations using AI-based image analysis. Alternative embodiments include a push-and-rotate cartridge and multiplexed detection panels for a number of biomarkers (e.g., ten biomarkers or thirty biomarkers).

In another aspect, the disclosed systems and methods exhibit significantly improved sensitivity (from 1.4±0.1 to 2.8±0.1 RU.mL/ng), reduced LOD (from 1.78±0.08 to 0.55±0.04 ng/mL), and expanded dynamic range (from 5-1000 to 1-1000 ng/mL) compared to conventional LFIAs, making them suitable for early disease diagnosis and home use by non-experts.

### BRIEF DESCRIPTION OF THE DRAWINGS

FIG. 1 depicts example embodiments of lateral flow immunoassay (LFIA) test strips;

FIG. 2 depicts a bar graph showing the colorimetric signal intensity (measured in relative units, RU) for the test strips depicted in FIG. 1;

FIG. 3 depicts the results of a flow dynamics simulation, illustrating velocity field disturbances;

FIG. 4 depicts example embodiments of LFIA test strips;

FIG. 5 depicts a bar graph showing the colorimetric signal intensity (measured in RU) for the test strips depicted in FIG. 4;

FIG. 6 depicts example embodiments of LFIA test strips;

FIG. 7 depicts an exploded view of an example embodiment of a two-step capillary blood-buffer cartridge;

FIG. 8 depicts an assembled view of an example embodiment of a two-step capillary blood-buffer cartridge;

FIG. 9 depicts an example embodiment of a process for using a two-step capillary blood-buffer cartridge;

FIG. 10 depicts example embodiments of capillary blood-buffer cartridges; and

FIG. 11 depicts an example embodiment of an algorithm for training an AI-based analyte quantification system.

### DETAILED DESCRIPTION OF THE EMBODIMENTS

Reference will now be made in detail to the present examples, including examples illustrated in the accompanying drawings. Wherever possible, the same reference numbers will be used throughout the drawings to refer to the same or like parts.

The present embodiments provide systems, devices, and methods for quantitatively detecting biomarkers using a geometrically enhanced lateral flow immunoassay (LFIA) platform for optional use with a finger-prick blood collection cartridge and AI-based detection. The invention overcomes limitations in conventional LFIA systems by improving sensitivity, enabling multiplexing, automating sample processing, and providing precise quantification through machine learning.

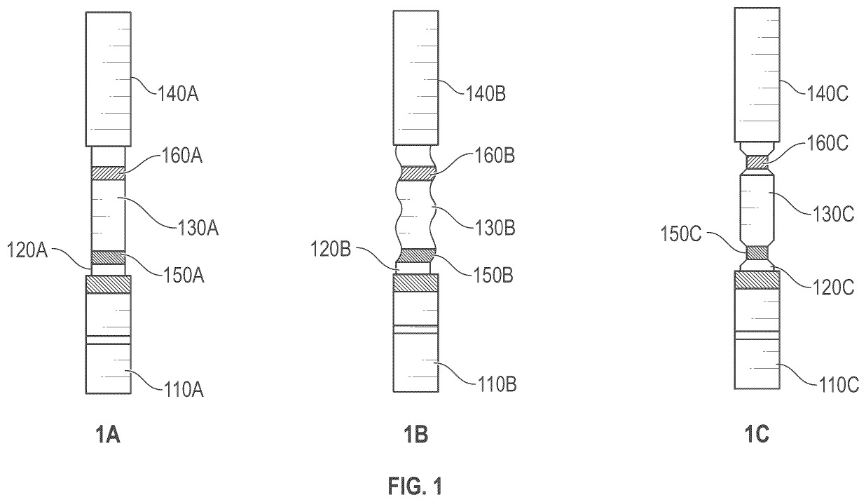

FIG. 1 depicts embodiments of LFIA test strips 1A, 1B, and 1C, each having its own geometric shape. In one aspect, each LFIA test strip depicted in FIG. 1 can include a sample pad 110, a conjugate pad 120, a NC membrane pad 130, and a wicking pad 140. In a further aspect, the NC membrane pad can include at least one test line (T-line) 150 and at least one control line (C-line) 160.

In another aspect, the LFIA test strips can be used in both sandwich-based and competitive assay formats, enabling detection of bio-analytes, minerals, large molecules (e.g., proteins), small molecules (e.g., vitamins, hormones, drugs), peptides, and miRNAs in various biofluids, including blood, tears, saliva, urine, and sweat. In use, the LFIA test strips can operate by leveraging capillary action to transport a biofluid sample through its components, facilitating the detection of target analytes in a sandwich or competitive assay format. When a biofluid sample is applied to the sample pad 110, it can be absorbed and can flow via capillary action to the conjugate pad 120, where nanoparticles, such as gold nanoparticles conjugated with recognition elements (e.g., antibodies or aptamers), bind to target analytes (e.g., antigens) in the sample. This analyte-recognition element complex can migrate to the NC membrane pad 130, where the T-line 150 can contain immobilized primary recognition elements (e.g., antibodies specific to the analyte) that can capture the complex, producing a visible colorimetric signal due to nanoparticle accumulation. The C-line 160, containing secondary recognition elements (e.g., anti-immunoglobulin antibodies), can capture excess conjugated nanoparticles to confirm assay validity, producing a second colorimetric signal. The wicking pad 140 drives continuous flow by absorbing excess sample, ensuring efficient migration through the strip.

In one aspect, the conjugate pad 120 can employ nanoparticles, such as gold, platinum, or quantum dots, in various suitable shapes (spheres, rods, wires, tubes, cubes, stars, dendrimers) and sizes. In some examples, recognition elements can include monoclonal and polyclonal antibodies, aptamers, and nanobodies, tailored for specific antigen interactions in sandwich or competitive assays.

In another aspect, the sample pad 110 can serve as the entry point for a sample, filtering and conditioning a biofluid to ensure consistent flow. The conjugate pad 120 can be designed to release nanoparticles uniformly, maximizing analyte binding efficiency. The NC membrane pad 130 can provide a stable platform for the T-line 150 and C-line 160, with its porosity and geometry influencing flow dynamics and interaction kinetics. The wicking pad 140 can maintain capillary flow, preventing backflow and ensuring complete assay completion within approximately six to fifteen minutes, in some examples. This integrated system, enhanced by geometric modifications and microfluidic elements, can achieve high sensitivity and specificity for quantitative biomarker detection across various biofluids, including blood, saliva, urine, tears, and sweat.

In some embodiments, the LFIA test strips can be integrated into a microfluidic circuit using medical-grade adhesives, including double-sided adhesive tapes for shaping different layers of the microfluidics system. In another aspect, the LFIA test strips described herein can include hydrophilic sheets to enhance capillary action. The hydrophilic sheets can cover the sample pad 110, conjugate pad 120, and NC membrane 140, for example, promoting uniform flow and mixing.

The LFIA test strips described herein can be assembled using a Kinbio XYZ Platform Dispenser (#HM3035) to apply clone 6 antibodies (1 mg/mL) to their respective T-line and goat anti-mouse IgG to their respective C-line at 1.5 μL/cm. AuNP-antibody solution can be dispensed onto the conjugate pad at 25 μL/cm. A CO2 laser cutter can be used to shape the NC membrane or conjugate pad for test strips comprising a non-conventional shape for those components (as described more fully below), while a Kinbio Automatic Strip Cutter (#ZQ2002) can cut straight strips (4 mm width) for more conventional designs. After drying for twenty-four hours in a low-humidity incubator, the sample pad, conjugate pad, and wicking pad can be glued to the NC membrane. Antigen-spiked samples (10-1000 ng/ml HBsAg in PBS with 0.05% NaN3, 0.05% Triton X100) can be tested by applying 200 μL to the sample pad with T-line (116) visibility after 15 minutes, as described in more detail below.

Each of test strips 1A, 1B, and 1C include a NC membrane pad 130 of unique geometry (compared to one another). Different geometries for the NC membrane pad 130 were tested to identify an optimal shape for enhancing antigen-antibody interactions and signal intensity, as measured at the T-line 150.

In one embodiment, test strip 1A comprises a conventional test strip having a standard rectangular NC member 130A to serve as a baseline in further testing. Test strip 1A can further include a sample pad 110A, a conjugate pad 120A, a wicking pad 140A, T-line 150A, and C-line 160A.

Test strips 1B and 1C comprise test strips having non-conventional and non-linear NC membrane pads. For example, test strip 1B can comprise a serpentine-shaped NC membrane pad 130B that can serve to increase the flow path length of the test strip and promote mixing of a sample with nanoparticles. Test strip 1B can further include a sample pad 110B, a conjugate pad 120B, a wicking pad 140B, T-line 150B, and C-line 160B.

In another embodiment, test strip 1C can include a NC membrane pad 130C having constriction zones located at the T-line 150C and the C-line 160C. The constriction zones can serve to focus flow and enhance signal intensity at the T-line and C-line. Test strip 1C can further include a sample pad 110C, a conjugate pad 120C, and a wicking pad 140C.

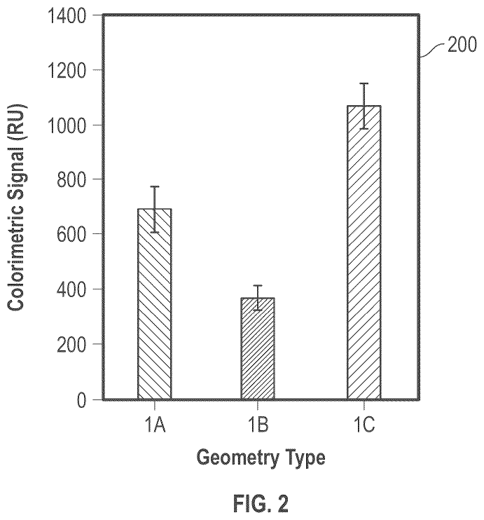

FIG. 2 depicts a bar graph 200 displaying results from testing conducted with respect to each of test strips 1A, 1B, and 1C using 200 μL spiked samples (500 ng/ml hepatitis B surface antigen, HBsAg). In one aspect, the testing showed that Test Strip 1C (i.e., the strip having constriction geometry) yielded significantly higher colorimetric signals (1069.9±81.1 relative units, RU) compared to Test Strip 1B (365.6±40.3 RU) and Test Strip 1A (690.4±83.3 RU).

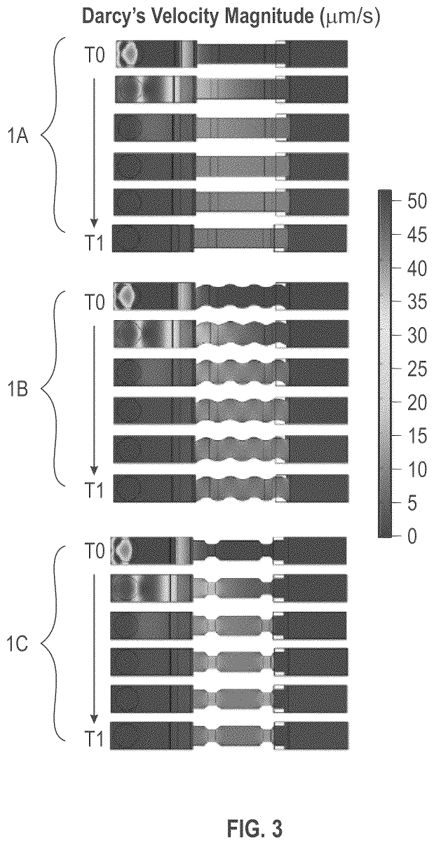

FIG. 3 depicts another aspect of testing performed with respect to each of test strips 1A, 1B, and 1C. As shown, numerical simulations using COMSOL Multiphysics 6.1 confirmed that the constriction zones in test strip 1C can disrupt flow velocity, prolonging antibody-antigen interaction time and increasing signal intensity. FIG. 3 depicts velocity field simulations over time intervals (e.g., from T₀ to T₁) for test strips 1A, 1B, and 1C, with constriction zones in Test Strip 1C corresponding to T-Line 150C and C-Line 160C showing enhanced flow disturbance. In the testing depicted in FIG. 3, T₀ was set to 1000 s and T₁ was set to 5000 s.

In some examples, COMSOL 6.1 Multiphysics can simulate flow dynamics using Richard's equation. FIG. 3 depicts field emission scanning electron microscopy (FESEM) images of the sample pads, conjugate pads, NC membrane pads, and wicking pads, with a filtered NC image used to calculate porosity via Python-based image processing (grayscale conversion, thresholding, Gaussian blur, median filter, morphological operations). Porosity and permeability can be derived, in some embodiments, informing simulation parameters (e.g., porosity ε, permeability k). A mesh of 858,196 tetrahedral elements and a 10⁻⁴ s time step can ensure accuracy. FIG. 3 depicts mesh and chemical reaction results for test strip 1A, 1B, and 1C geometries, respectively, showing higher T-line interactions for test strip 1C. The simulation can further use a 150 μL inlet mass flow rate (48.7E-6 kg/s) over 3.08 s, with initial pressure at 0 Pa, in some embodiments.

Accordingly, the flow velocity testing supports a conclusion that a test strip 1C having constriction zones located at its respective T-line 150C and C-line 160C performs better, in terms of signal intensity, than conventional rectangular test strips.

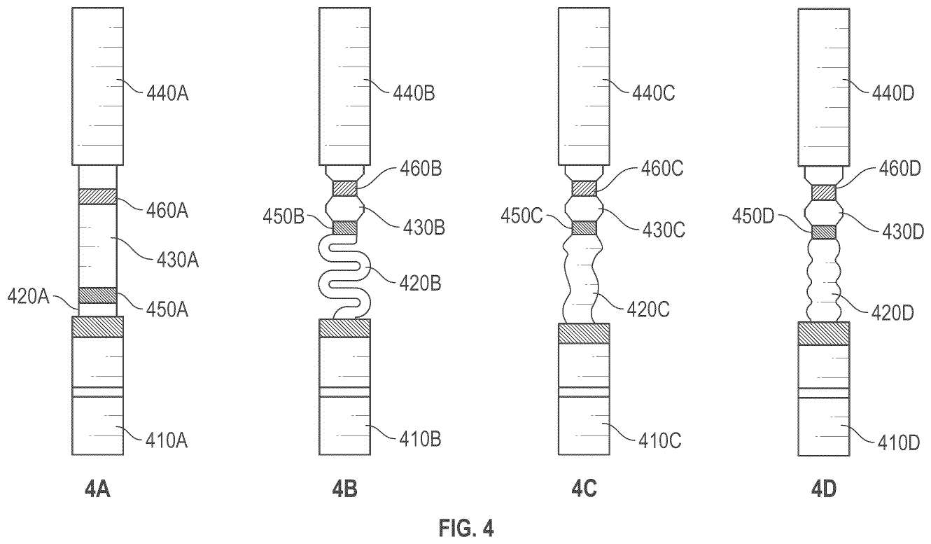

FIG. 4 depicts further embodiments of LFIA test strips, each having constriction zones located at their respective T-lines 450 and C-lines 460, but each having a uniquely shaped (with respect to one another) conjugate pad 420. In one aspect, the conjugate pads 420 can be made from glass fiber conjugate 10 mm×300 mm strips. Test strips 4B-4D can comprise geometrically modified patterns to enhance mixing of recognition elements with target antigens. These patterns can include, but are not limited to, meandering, serpentine, sinusoidal, spiral, zig-zag, and nozzle-diffuser designs, one or more of which can increase shear stress and promote incubation of secondary antibody (Ab)-conjugated gold nanoparticles (AuNPs, 114) with antigens.

LFIA test strip 4A can comprise a conventional test strip having a standard rectangular conjugate pad 420A and a conventionally shaped sample pad 410A, NC member 430A, wicking pad 440A, T-line 450A, and C-line 460A to serve as a baseline in further testing.

Test strip 4B comprises an alternative embodiment including an NC membrane 430B having constriction zones located at its T-line 450B and C-line 460B, similar to the test strip 1C in FIG. 1, but test strip 4B further includes a meandering conjugate pad 420B upstream from its NC membrane pad 430B. Test strip 4B can further include a sample pad 410B and a wicking pad 440B.

In another embodiment, test strip 4C can include a NC membrane pad 430C with constriction zones substantially similar to test strips 4B and 1C, but further including a sinusoidal conjugate pad 420C. Test strip 4C can further include a sample pad 410C and a wicking pad 440C.

In still another embodiment, test strip 4D can include a NC membrane pad 430D with constriction zones but further including a diffuser-nozzle conjugate pad 420D. Test strip 4D can further include a sample pad 410D and a wicking pad 440D.

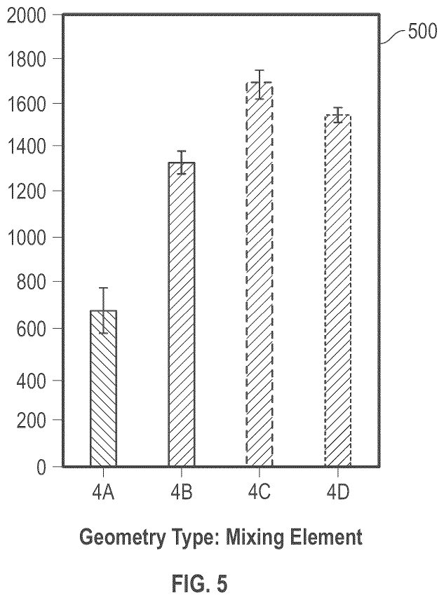

FIG. 5 depicts results from testing conducted with respect to each of LFIA test strips 4A, 4B, 4C, and 4D using a bar graph 500. In one aspect, testing was performed using the same 200 μL spiked samples (500 ng/ml hepatitis B surface antigen, HBsAg) as was used in the testing depicted in FIGS. 2 and 3. As shown, the testing demonstrated that test strip 4C (having a sinusoidal conjugate pad 420C) produced the highest signal intensity of the four strips tested, measuring 1693.3±69.8 RU. In another aspect, test strip 4D (having a diffuser-nozzle shaped conjugate pad 420D) produced the second highest signal intensity at 1548.4±31.9 RU, followed by test strip 4B (having a meandering conjugate pad 420B) with a signal intensity of 1344.2±49.5 RU. Test strip 4A, which is the conventionally shaped test strip having a substantially rectangular conjugate pad 420A and NC membrane pad 430A performed the worst of the four samples in the testing of signal intensities.

Further testing (not depicted) was also performed with respect to assay timing for each of the four test strips 4A, 4B, 4C, and 4D. In one aspect, that testing demonstrated that test strips 4C and 4D completed in 5.9±0.5 min and 5.6±0.4 min, respectively. In another aspect, test strip 4A (the conventional test strip) completed in 7.5±0.4 min and test strip 4B completed in over 15 min. COMSOL simulations substantially similar to the tests depicted in FIG. 3 were also performed with respect to test strips 4A, 4B, 4C, and 4D. Those simulations confirmed that the sinusoidal conjugate pad 420C of test strip 4C can enhance flow disturbance and mixing, and can improve antigen-antibody interactions with respect to a conventionally shaped, rectangular test strip.

In additional testing, test strip 4C (having the sinusoidal conjugate pad 420C design) achieved a limit of detection (LOD) of 0.55±0.04 ng/mL for HBsAg. The LOD for test strip 4C represented a threefold improvement over the conventional test strip 4A which achieved a LOD of 1.78±0.08 ng/mL. Analytical sensitivity also increased from 1.4±0.1 for the conventional test strip 4A to 2.8±0.1 RU.mL/ng for test strip 4C. In a further aspect, dynamic range was also improved, expanding from 5-1000 ng/mL for the conventional test strip 4A to 1-1000 ng/mL for test strip 4C, allowing for greater flexibility in quantification across a wider concentration range. Specificity associated with the test strips was confirmed using control proteins (BSA, CRP, SARS-COV-2 N-protein, S-protein), with only HBsAg producing a T-line signal.

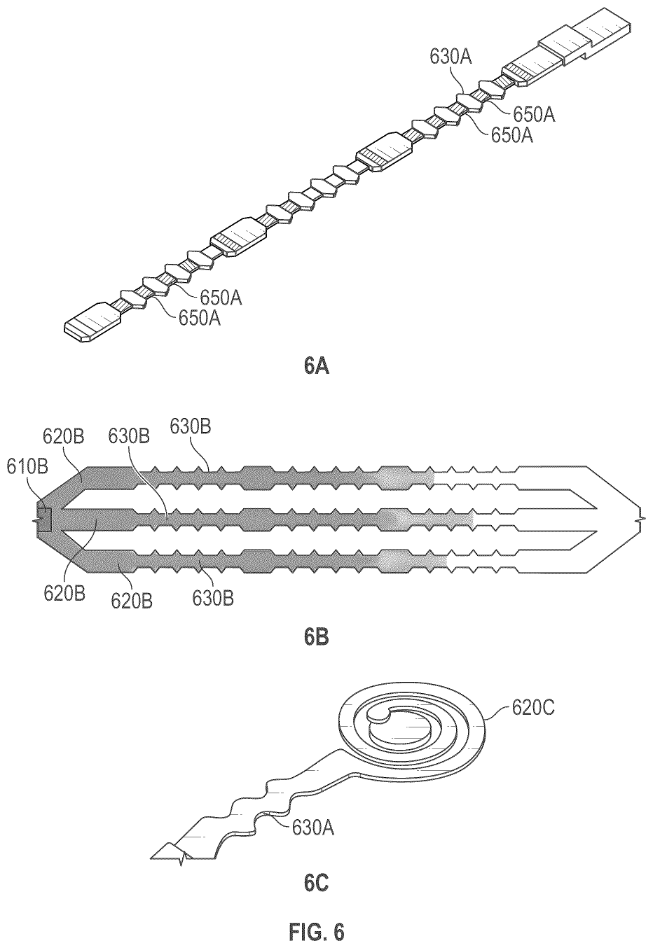

In addition to the various shapes of NC membrane pads and conjugate pads associated with linear test strips having a single T-line and a single C-line, FIG. 6 depicts alternative embodiments of LFIA test strips comprising multiple, serial testing zones (6A), testing strips comprising a plurality of parallel testing zones (6B), and testing strips comprising other conjugate pad shapes (6C). In one aspect, the test strips 6A, 6B, and 6C can be fabricated using CO2 laser cutting for precise shaping of their respective conjugate pads and NC membrane pads.

In some embodiments, test strip 6A comprises a number of serial, or sequential, T-lines 650A, each at a respective constriction zone along a NC membrane pad 630A. The serial T-lines 650A can be used to quantify the detection of multiple biomarkers. For example, as a sample flows along NC membrane pad 630A and passes through each sequential T-line 650A, the colorimetric signal intensity decreases. The greater the concentration of a target analyte in a sample, the further down the test strip a T-line will indicate a strong colorimetric signal. In this way, a concentration or amount of the target analyte in a sample can be quantified.

Test strip 6B comprises an alternative test strip embodiment 6B comprising a plurality of parallel testing zones for detecting more than one target analyte. For example, in some embodiments, test strip 6B can comprise a sample pad 610B that can wick to three parallel conjugate pads 620B. In a conventional test strip, the conjugate pad can vary depending on the target analyte to be detected. The conjugate pad of a test strip is typically impregnated with detection reagents, such as antibodies, antigens, or other binding molecules, that are conjugated to a signal-generating particle (e.g., gold nanoparticles, latex beads, or fluorescent tags). These reagents are chosen based on their specificity to the target analyte. For example, for detecting a protein biomarker, the conjugate might include antibodies specific to that protein. In another example, for detecting a small molecule (e.g., a drug), a hapten-specific antibody or antigen conjugate may be used. Alternatively, for nucleic acid detection, the conjugate could involve labeled DNA or RNA probes.

In one aspect, the three parallel and independent conjugate pads 620B of test strip 6B, each wicking from sample pad 610B, can comprise its own detection reagent(s) for detecting a unique (with respect to the other conjugate pads 620B) target analyte. In this way, multiple target analytes can be detected simultaneously.

In further embodiments, the concepts of test strip 6A having a number of sequential or serial T-lines positioned along an elongated NC membrane pad can be used in conjunction with the parallel conjugate pads 620B of test strip 6B, resulting in a test strip that can not only simultaneously detect the presence of multiple target analytes but can also quantify the concentration or amount of each target analyte in a sample. For example, as depicted by test strip 6B in FIG. 6, the colorimetric signal intensity indicated by each sequential T-line of parallel conjugate pads 620B diminishes in a way that the concentration or amount of a target analyte in the sample can be quantified.

It should be noted that the number of serial T-lines depicted in FIG. 6 with respect to test strip 6A and/or the number of parallel testing zones depicted in FIG. 6 with respect to test strip 6B are exemplary and only illustrative of some possibilities. Test strips having more or fewer sequential T-lines and/or more or fewer parallel testing zones are also possible, provided the sample size and the wicking functionality of the sample pad, conjugate pad, NC membrane pad, and wicking pad can facilitate adequate transference of the sample along the test strip. In some embodiments, testing strips employing one or both of the sequential T-lines and parallel testing zones of test strips 6A and 6B, respectively, can be used for detection of up to at least thirty biomarkers (e.g., a number X of target analytes multiplied by a number Y of sequential T-line quantification measurements for each target analyte).

In still another embodiment, test strip 6C of FIG. 6 comprises a spiral-shaped conjugate pad 620C upstream of a NC membrane pad 630C. In such embodiments, the longer mixing time of a sample flowing through conjugate pad 620C can enhance sample mixing and flow control. In another aspect, the design is also more compact than a more linear design that would extend the overall length of the test strip.

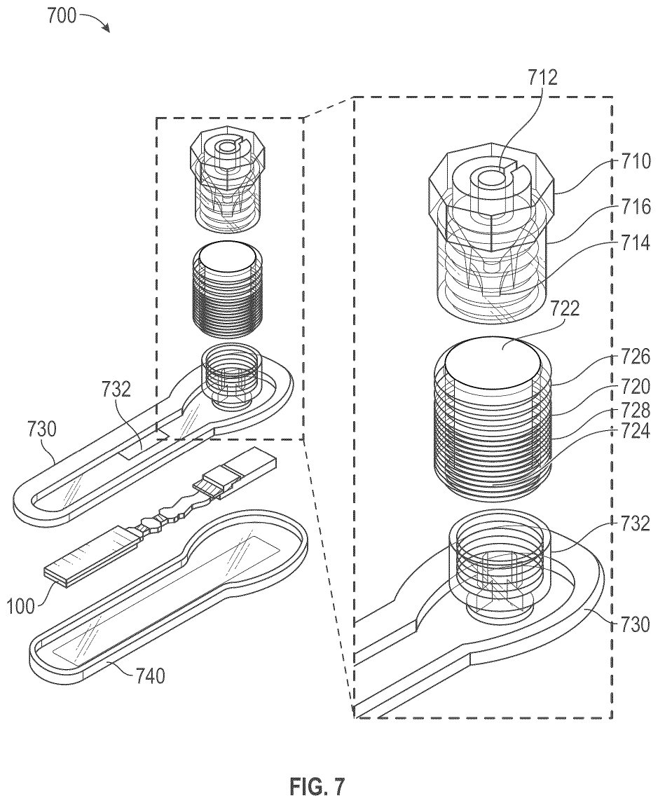

FIG. 7 depicts a capillary blood-buffer cartridge 700 having a two-step rotation mechanism, in accordance with some embodiments. The cartridge 700 includes a capillary blood housing 710, a buffer capsule 720, a cartridge lid 730 and a cartridge base 740. LFIA test strips 100 can be housed within the cartridge 700 for automated sample-to-result detection. Test strips 100 can be, for example, any of the test strips described above with respect to other embodiments. In one aspect, the cartridge 700 can simplify blood collection, buffer mixing, and sample application to a test strip, making it suitable for non-expert users.

In some embodiments, the capillary blood housing 710 can include a capillary blood collection chip 712 comprising medical-grade adhesives and hydrophilic sides, allowing for aliquoting a specific volume of finger prick blood (e.g, 5-10 μL) into the chip. For example, the housing 710 can include EDTA-coated capillary tubes to prevent coagulation and a sharp tip 714. As depicted in FIG. 7, the housing 710 can further include female threading 716 of a first pitch for mating with male threading of the buffer capsule 720.

In another aspect, the buffer capsule 720 can store an antigen buffer (e.g., PBS with 0.05% NaN₃, 0.05% Triton X-100) sealed with a top sheet of aluminum foil 722 and a bottom sheet of aluminum foil 724. The buffer capsule 720, in some embodiments, includes two different thread structures, one on each of its ends. For example, as depicted in FIG. 7, the buffer capsule can comprise male threading 726 of a first pitch on its outer surface and at its upper end (i.e., the end that can couple to the housing 710) and male threading 728 of a second pitch on its outer surface and at its lower end (i.e., the end that can couple to the cartridge lid 730). It should be appreciated that the threading of buffer capsule 720 can be male or female, provided the upper end of the capsule 720 can mate with the housing 710 and the lower end of the capsule 720 can mate with the cartridge lid 730.

In a further aspect, the cartridge lid 730 can comprise a female threaded opening 732 having the second pitch corresponding to the second pitch of the threading 728 of buffer capsule 720. The cartridge lid 730 is further configured to couple to the cartridge base 740 and house a test strip 100 therebetween. The test strip 100 can be housed between the cartridge lid 730 and the cartridge base 740 is such a way that the sample pad of test strip 100 is located just below the opening 732 of the cartridge lid 730. In some embodiments, the cartridge lid 730 can further comprise a sharp projection extending toward the lower end of the buffer capsule 720 for piercing the aluminum foil sheet 724 of the buffer capsule.

As described above with respect to other embodiments, the test strip 100 can include a sample pad 110, a conjugate pad 120, a NC membrane pad 130 with one or more T-lines and at least one C-line, and a wicking pad 140.

In some examples, the cartridge 700, including any or all of its components, can be fabricated using 3D printing (e.g., Formlabs 3B printer using clear/white resins). The cartridge 700 can then be washed with 2-propanol and cured at 60° C. for 30 minutes, in some embodiments.

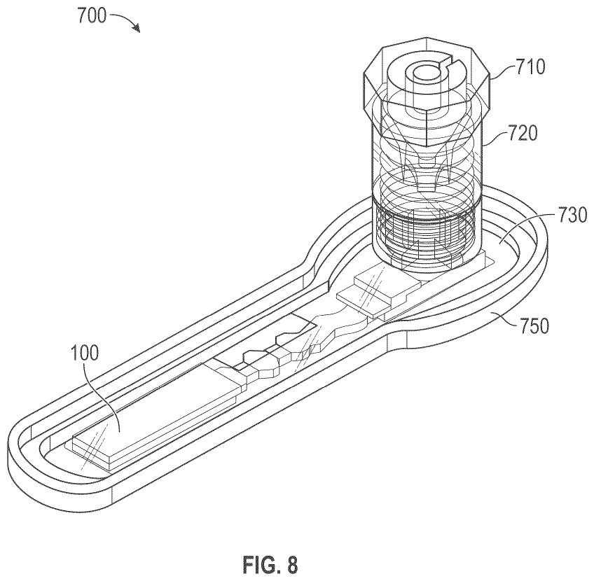

FIG. 8 depicts an assembled cartridge 700 housing a test strip 100, in some examples. As shown, test strip 100 can be placed and encapsulated between the cartridge base 740 and the cartridge lid 730, such that the sample pad 110 of test strip 100 is located under the threaded opening 732 of the cartridge lid 730.

In another aspect, the buffer capsule 720 can be threadedly coupled to the cartridge lid 730 at the capsule's lower end by mating the threads 728 of the capsule 720 with the threads 732 of the cartridge lid 730. The buffer capsule 720 can also be threadedly coupled to the capillary blood housing 710 at the capsule's upper end by mating threads 726 of the capsule 720 with the threads 716 of the housing 710. In some embodiments, the mating threads 726 and 716 of the capsule 720 and the housing 710, respectively, are of a first pitch, and the threads 728 and 732 of the capsule 720 and lid 730, respectively, are of a second pitch. In further embodiments, the different pitches of the threading on opposing ends of the capsule 720 facilitates a two-step rotation mechanism because the different pitches result in different shear stresses between the threads and, as a result, different torques are required for rotation. In some embodiments, the pitch of threads 726 and 716 is greater or larger than the pitch of threads 728 and 732. In this way, a first relatively easier rotation (i.e., a rotation requiring less force than the second rotation described below) of housing 710 on to capsule 720 can cause sharp tip 714 of housing 710 to pierce the aluminum foil sheet 722 of the buffer capsule 720 such that blood contained in the housing 710 enters the previously encapsulated buffer contained in the capsule 720 and mixes with the buffer. In further embodiments, once blood enters the capsule 720, the capsule 720 can be shaken or agitated (e.g., by hand or by machine) to ensure sufficient mixing of the blood and the buffer.

In another aspect, once the blood sample is mixed with the buffer, the housing 110 and buffer capsule 720 can be further rotated along the smaller or finer pitch of threads 728 and 732 (i.e., smaller or finer relative to the pitch of threads 716 and 726). This second rotation is relatively harder (i.e., requires greater force) than the first rotation described above, providing a user with tactile and intuitive feedback with respect to where the first step in the two-step rotation ends and where the second step in the two-step rotation begins. In some embodiments, upon the second-step rotation, the sharp projection of the cartridge lid 730 can pierce the aluminum foil sheet 724 at the lower end of the capsule 720, which can allow the blood and buffer mixture to enter the opening in the cartridge lid and contact sample pad 110 of test strip 100.

In a further aspect, the blood sample can flow from the sample pad 110, to the conjugate pad 120, NC membrane pad 130, and wicking pad 140 of testing strip 100, as described previously with respect to other embodiments. In some examples, cartridge lid 730 can further include an opening or window 732 that exposes the T-line(s) and/or C-line(s) of test strip 100 for easy viewing by a user.

During testing, 50 μL of blood spiked with 1 μg/mL HBsAg confirmed reliable performance of the cartridge 700. In some embodiments, blood separator fibers (e.g., two layers of Fusion 5) were optimized to capture red blood cells, reducing interference with AuNP red coloration. The testing confirmed optimal performance with Fusion 5 over LF1 or no separator.

In other embodiments, an alternative cartridge employing a “push- and rotate” mechanism with a spring can be used instead of the two-step rotation described above with respect to cartridge 700 described above. In some such embodiments, the capillary blood housing 710 can be pushed (via spring or sliding) to rupture the top aluminum foil 722 of the buffer capsule 720, thereby transferring blood into the buffer contained in the capsule 720. In one aspect, after mixing, shaking, or agitating the capsule 720 to mix the blood with the buffer, a rotation of the capsule 720 with respect to the cartridge lid 730 (e.g., rotation with respect to previously-described threads 728 and 732 of the capsule and lid, respectively) can rupture the aluminum foil sheet 724 of the capsule 720 via a sharp projection extending upward from the cartridge lid 730, delivering the blood mixture to the sample pad 110 of the test strip 100 contained between cartridge lid 730 and cartridge base 740.

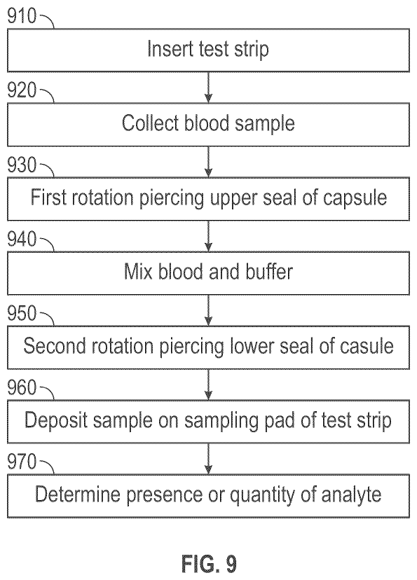

FIG. 9 depicts a flowchart of steps, in some embodiments, performed in the operation of cartridge 700. At step 910, a test strip having a sample pad, a conjugate pad, a NC membrane pad, and a wicking pad can be placed within the cartridge between its lid 730 and base 740. At step 920, a blood sample can be introduced into the housing 710. The sample can be collected via a finger prick or any other suitable manner, such as extracted from a test tube. At step 930, a user can rotate housing 710 with respect to buffer capsule 720 to cause the piercing of the seal (e.g., aluminum foil sheet) 722 on the upper surface of the capsule 720. The blood sample can then flow into the capsule 720 and mix with the buffer therein.

At step 940, the blood and buffer can be mixed by shaking or agitating (e.g., by hand or by machine) the cartridge 700. Once sufficiently mixed, the user can rotate the housing 710 and capsule 720 with respect to the cartridge lid 730 at step 950 to cause the piercing of the seal (e.g., aluminum foil sheet) 724 on the lower surface of the capsule 720.

At step 960, the blood and buffer mixture can flow to the sample pad of the test strip and wick across the length of the test strip along the conjugate pad, the NC membrane pad, and the wicking pad. During the wicking process, the mixture passes through one or more T-lines and/or C-lines. At step 970, the resulting colorimetric signal intensity can be used to determine the presence and/or quantity of a target analyte.

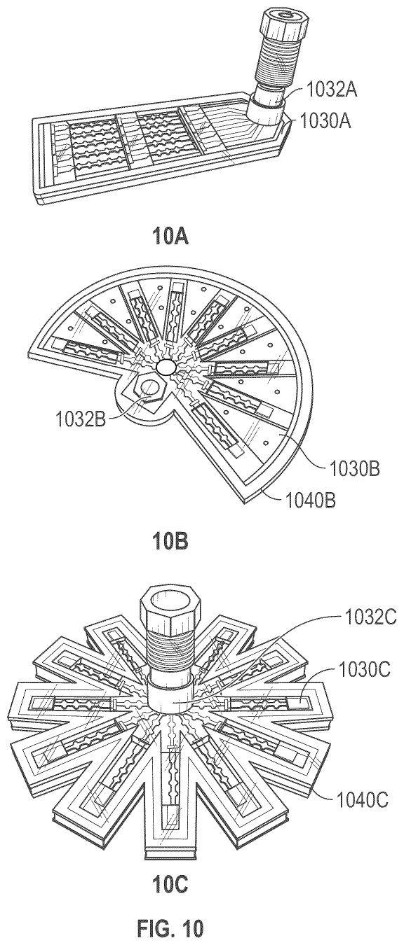

FIG. 10 depicts alternative embodiments 10A, 10B, and 10C of a capillary blood-buffer cartridge. In one aspect, any one or more of cartridges 10A, 10B, and 10C can be two-step rotation mechanisms (such as the cartridge depicted in FIGS. 7 and 8) or push-and-rotate mechanisms (described previously). In another aspect, the cartridges 10A, 10B, and 10C are configured for use with one or more of the LFIA test strips described above with respect to FIG. 6, as just some examples.

For example, cartridge 10A can comprise the two-step rotation mechanism described with respect to cartridge 700 and further comprise a cartridge lid 1030A and cartridge base 1040A configured to accommodate a wider testing strip having multiple parallel testing zones, similar to the testing strip 6B described above with respect to FIG. 6. In such embodiments, the test strip 6B can be positioned between the cartridge lid 1030A and cartridge base 1040A such that the sample pad 610B is located just beneath the threaded opening 1032A of the cartridge 10A.

In another example, cartridge 10B can be a semi-circular design configured to accommodate a semi-circular test strip having a plurality of radially extending testing zones. In such embodiments, the semi-circular test strip can be positioned between the cartridge lid 1030B and cartridge base 1040B such that a sample pad of the semi-circular test strip is located just beneath the threaded opening 1032B of the cartridge 10B.

In a further example, cartridge 10C can be a circular design configured to accommodate a circular test strip having a plurality of radially extending testing zones. In such embodiments, the circular test strip can be positioned between the cartridge lid 1030C and cartridge base 1040C such that a central sample pad of the circular test strip is located beneath a centrally-located threaded opening 1032C of the cartridge 10C.

In another aspect, one or both of the cartridge lid and cartridge base of any cartridge embodiment described herein can have recesses or channels in their interior surfaces intended to receive a test strip having a shape corresponding to the cartridge. Such recesses or channels can ensure, in some embodiments, that the sample pad of the test strip is positioned properly within the cartridge and the test strip does not shift or move within the cartridge during testing.

It should be further appreciated that any one or more of the test strip and cartridge designs described with respect to FIG. 10 can include a plurality of sequential T-lines and/or C-lines along each of their testing zones, as described above with respect to testing strip 6A of FIG. 6.

In a further aspect, an AI-based detection system can be used in conjunction with the aforementioned systems to quantify biomarker concentrations. In some embodiments, the AI-based systems can comprise a processor, memory, a machine learning algorithm, and a scanner (e.g., an Epson WorkForce DS-50000 scanner).

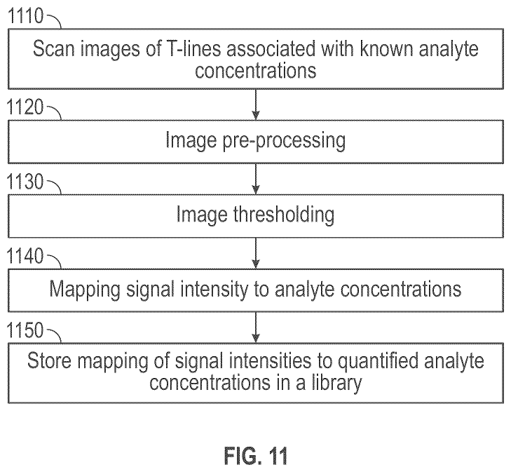

FIG. 11 depicts a flowchart depicting an example training algorithm for the the AI-based analyte quantification system. At step 1110, the scanner can capture images of T-lines from test strips corresponding to known and specific concentrations of an analyte for the purpose of training the system. During the training process, at step 1120, the algorithm can receive the scanned images as inputs and perform image pre-processing steps including, for example, pre-processing steps intended to reduce noise or enhance the features of an image captured by the scanner. For example, in some embodiments, the algorithm can use Python-based processing, such as Gaussian blur to reduce noise followed by grayscale conversion. At step 1130, the algorithm can perform one or more thresholding steps that can, for example, segment images captured by the scanner by converting the images into a binary or simplified form based on pixel intensity values, in some examples. In some embodiments, the thresholding step(s) can include using Otsu's algorithm to isolate colorimetric signals. At step 1140, the algorithm can then perform signal analysis by, in some examples, using a density estimator that can map or associate a detected signal intensity to the known analyte concentration (e.g., 1-1000 ng/ml HBsAg). At step 1150, the resulting mapping and associations between detected colorimetric signal intensities and quantified analyte concentrations can then be stored in a library for future recall and cross-referencing.

Once the AI-based detection system is trained in accordance with the foregoing steps, an image of a T-line of a test strip with an unknown analyte concentration level can be captured and input into the system, and the system can return a quantified concentration level of the analyte by mapping the detected colorimetric signal intensity of the captured image to a corresponding analyte concentration from the library of stored data.

What is claimed is:

1. A lateral flow immunoassay (LFIA) test strip, comprising:
a sample pad configured to receive a biological sample;
at least one conjugate pad having a geometrically non-linear shape;
at least one nitrocellulose (NC) membrane pad having at least one test line and at least one control line, the test line being positioned within a constriction zone;
at least one wicking pad configured to drive capillary flow.

2. The LFIA test strip of claim 1, wherein the non-linear shape of the at least one conjugate pad is a sinusoidal, meandering, serpentine, spiral, zig-zag, or nozzle-diffuser shape.

3. The LFIA test strip of claim 1, wherein the NC membrane pad includes a plurality of test lines along its longitudinal extension.

4. The LFIA test strip of claim 1, wherein the at least one NC membrane pad is a plurality of NC membrane pads, each NC membrane pad in fluid communication with the sample pad.

5. The LFIA test strip of claim 4, wherein each of the plurality of NC membrane pads extend in a direction that is parallel with respect to the other NC membrane pads.

6. The LFIA test strip of claim 4, wherein each of the plurality of NC membrane pads extend outward from the sample pad radially.

7. The LFIA test strip of claim 1, wherein the at least one NC membrane pad comprises a microfluidic circuit that includes fibers and one or more double-sided adhesive tapes for shaping a plurality of layers.

8. A capillary blood-buffer cartridge configured for receiving a lateral flow immunoassay (LFIA) test strip, the cartridge comprising:
a blood housing 710;
a capsule encapsulating a buffer, the capsule having a first end configured for coupling to the blood housing and a second end configured for coupling to a cartridge lid; and
a cartridge base configured to couple to the cartridge lid, defining a cavity between the cartridge lid and the cartridge base.

9. The capillary blood-buffer cartridge of claim 8, wherein the first end of the capsule comprises threading of a first pitch, and the second end of the capsule comprises threading of a second pitch, the second pitch being smaller than the first pitch.

10. The capillary blood-buffer cartridge of claim 8, wherein the capsule comprises an upper seal at its first end and a lower seal at its second end.

11. The capillary blood-buffer cartridge of claim 8, wherein the blood housing comprises a tip configured to pierce the upper seal of the first end of the buffer capsule.

12. The capillary blood-buffer cartridge of claim 8, wherein the cartridge lid comprises a tip configured to pierce the lower seal of the second end of the buffer capsule.

13. The capillary blood-buffer cartridge of claim 8, wherein the cavity defined between the cartridge lid and the cartridge base is shaped to receive a LFIA test strip.

14. The capillary blood-buffer cartridge of claim 13, wherein the cartridge lid comprises an aperture for viewing the test line of the LFIA test strip.

15. An AI-based training system for use in quantifying colorimetric signal intensities associated with lateral flow immunoassay (LFIA) test strips, the training system comprising:
a plurality of LFIA test strips, each of the LFIA test strips comprising a nitrocellulose membrane portion that includes a test line exposed to a sample containing a known concentration of a target analyte;
a scanner that captures an image of the test line for each of the LFIA test strips;
a processor configured to:
perform one or more pre-processing steps to reduce noise in each of the images captured by the scanner;
determining a colorimetric signal intensity associated with each of the images;
for each of the images, associating the determined colorimetric signal intensity with the known concentration of the target analyte; and
a database that stores the associations between the determined colorimetric signal intensities with the known concentrations of the target analyte for the purpose of quantifying concentrations of the target analyte present at the test line of a LFIA test strip exposed to a sample containing an unknown concentration of the target analyte.

16. The AI-based training system of claim 15, wherein the pre-processing steps include using a Gaussian blur technique with respect to each image.

17. The AI-based training system of claim 15, wherein determining a colorimetric signal intensity associated with each of the images includes a thresholding step.

18. The AI-based training system of claim 17, wherein the thresholding step includes using Otsu's algorithm with respect to each image.

19. The AI-based training system of claim 15, wherein the test line of each of the plurality of LFIA test strips is located in a respective constriction zone.

20. The AI-based training system of claim 15, wherein each of the plurality of LFIA test strips further comprises a conjugate pad having a non-linear shape.
